(19) United States
(12) Patent Application Publication
(10) Pub. No.: US 2024/0219371 A1
(43) Pub. Date: Jul. 4, 2024

(54) APPARATUS FOR CONDUCTING URINALYSIS

(71) Applicant: Vivosens Inc, San Francisco, CA (US)

(72) Inventors: Miray TAYFUN, San Francisco, CA (US); Balim Bengisu Caf, Istanbul (TR); Sahin Cakan, Istanbul (TR); Emre Gokturk, Istanbul (TR)

(21) Appl. No.: 18/149,110

(22) Filed: Jan. 1, 2023

(51) Int. Cl. G01N 33/493; B01L 3/00

(52) U.S. Cl. CPC G01N 33/493; B01L 3/502; B01L 2200/04; B01L 2200/0689; B01L 2300/0609; B01L 2300/0663; B01L 2300/0825; B01L 2300/0848; B01L 2400/049

(57) ABSTRACT

An apparatus for conducting urinalysis of urine is provided. The apparatus includes a housing mountable on a urination device defining a urination area; a tray for holding a plurality of cartridges, each of which including a urine strip having testing pads, the tray is movable inside the housing and is configured to displace at least one cartridge toward an extracting spot; a dispenser configured for extracting the urine strip out of the cartridge and conveying the testing pads into the urination area along an extraction path extending between the extracting spot and the urination area; and a detector configured to obtain data from the testing pads when the testing pads are transferred along the extraction path.

### FIELD OF INVENTION

The presently disclosed subject matter relates to an apparatus for conducting urinalysis.

### BACKGROUND

Urinalysis is the physical, chemical, and microscopic examination of urine. It involves several tests to detect and measure various compounds that pass through the urine. When done on a frequent basis, urinalysis allows tracking of changes in a person's body chemistry on a day-to-day basis.

### SUMMARY OF INVENTION

There is provided in accordance with an aspect of the presently disclosed subject matter an apparatus for conducting urinalysis of urine. The apparatus includes a housing mountable on a urination device defining a urination area; a tray for holding a plurality of cartridges, each of which including a urine strip having testing pads, the tray is movable inside the housing and is configured to displace at least one cartridge toward an extracting spot; a dispenser configured for extracting the urine strip out of the cartridge and conveying the testing pads into the urination area along an extraction path extending between the extracting spot and the urination area; and a detector configured to obtain data from the testing pads when the testing pads are transferred along the extraction path.

The dispenser can include a spout defining a channel extending between the extracting spot inside the housing and the urination area.

The tray can be movable along a sliding axis, wherein the sliding axis is perpendicular to the extraction path, and extend between a first side of the dispenser and a second side of the dispenser.

The dispenser can include an extractor configured to engage the cartridge and to selectively rotate in a first direction and a second direction, wherein rotation in the first direction urges the urine strip to be extracted out of the cartridge and rotation in the second direction urges the urine strip to be retracted back into the cartridge.

The cartridge can include a case and a reel rotatably mounted inside the case, the reel is configured to allow winding the urine strip thereon, and is configured such that rotation thereof extracts the urine strip out of the case.

The reel can include an aperture and a plurality of gear teeth, and the extractor includes a cogwheel configured to engage the plurality of gear teeth and to rotate the reel about an axis perpendicular to the extraction path.

The apparatus can further include a compartment configured to hold used cartridge, the compartment includes an opening disposed adjacent the extracting spot.

The apparatus can further include a discarding member movable with respect to the extracting spot between a forward position and a backward position, wherein in the forward position a horizontal portion of the discarding member covers the opening of the compartment, and wherein in the backward position the opening allows discarding the cartridge from the tray into the compartment.

The discarding member can include a vertical portion configured such that in the forward position the vertical portion protrudes into the compartment, pushing thereby cartridges disposed below the opening toward an inner section of the compartment.

The dispenser can define a testing spot along the extracting axis and the detector is disposed at the testing spot.

The testing spot can be defined inside the and the detector is configured to obtain an image of the testing pads while the urine strip is retracted back into the cartridge.

The dispenser can include a seal configured to displace in a first direction in which the seal is urged into the channel and a second direction in which the seal is displaced out of the channel allowing the urine strip to move through the channel.

The apparatus can further include a cleaning mechanism configured for clearing the channel of any residues of urine.

The cleaning mechanism can include an air pump configured to generate an airflow inside the channel forcing any remaining urine out of the channel towards the urination area.

The air pump can be further configured to suck air from an inner space of the housing creating thereby vacuum in the housing.

The channel can include a hydrophobic surface, repelling any remaining urine.

The apparatus can further include a mounting member configured to mount the housing to an outer wall of a urination device.

The mounting member can include a seat for holding a corresponding flange defined on the housing.

The mounting member can include a bottom arm configured to be affixed to the outer wall, and a least one top arm configured to be affixed to a top lip of the urination device.

The mounting member can further include an adjusting pivot configured for adjusting an angle between the bottom arm and the at least one top arm.

### BRIEF DESCRIPTION OF THE DRAWINGS

In order to understand the disclosure and to see how it may be carried out in practice, embodiments will now be described, by way of non-limiting examples only, with reference to the accompanying drawings, in which:

FIG. 1 is a side perspective view of the apparatus for conducting urinalysis in accordance with an example of the presently disclosed subject matter;

FIG. 2A is a partially exploded front view of the apparatus of FIG. 1;

FIG. 2B is a partially exploded back view of the apparatus of FIG. 1;

FIG. 2C is a partially exploded back view of the apparatus of FIG. 1, with the compartment removed;

FIG. 3A is a back view of the inner elements of the apparatus of FIG. 1, with the housing removed showing the tray in a first position;

FIG. 3B is a back view of the inner elements of FIG. 3A, with the housing removed showing the tray in a second position;

FIG. 3C is a sectional view of the tray and compartment of the apparatus of FIG. 1 showing the discarding member in a forward position;

FIG. 3D is a sectional view of the tray and compartment of the apparatus of FIG. 1 showing the discarding member in a backward position;

FIG. 3E is a sectional view of the tray and compartment of the apparatus of FIG. 1 showing the discarding member in a forward position with a used cartridge;

FIG. 3F is a partial cutaway view of the tray and compartment of the apparatus of FIG. 1;

FIG. 4A is a side perspective view of a cartridge in accordance with an example of the presently disclosed subject matter;

FIG. 4B is a side perspective view of the cartridge of FIG. 4A, with a urine strip extracted;

FIG. 4C is a side view of the cartridge of FIG. 4B;

FIG. 5A is a sectional view of the apparatus of FIG. 1 showing a urine strip extracted;

FIG. 5B is a sectional view of the apparatus of FIG. 1;

FIG. 6 is a partially exploded back view of the apparatus of FIG. 1, showing the discarding member and cleaning mechanism;

FIG. 7A is a perspective view of the toilet having the apparatus of FIG. 1 mounted thereon;

FIG. 7B is a side sectional view of the toilet of FIG. 7A; and

FIG. 7C is a perspective view of a mounting member in accordance with an example of the presently disclosed subject matter.

### DETAILED DESCRIPTION OF EMBODIMENTS

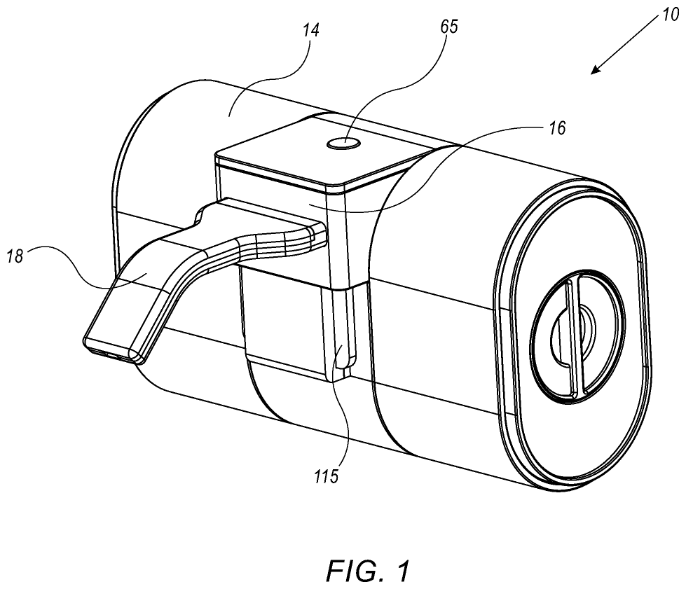

FIG. 1 shows an apparatus 10 for conducting urinalysis, including a mountable housing 14, for mounting on a urination device defining urination area, such as a toilet, urinal, female urinal etc.

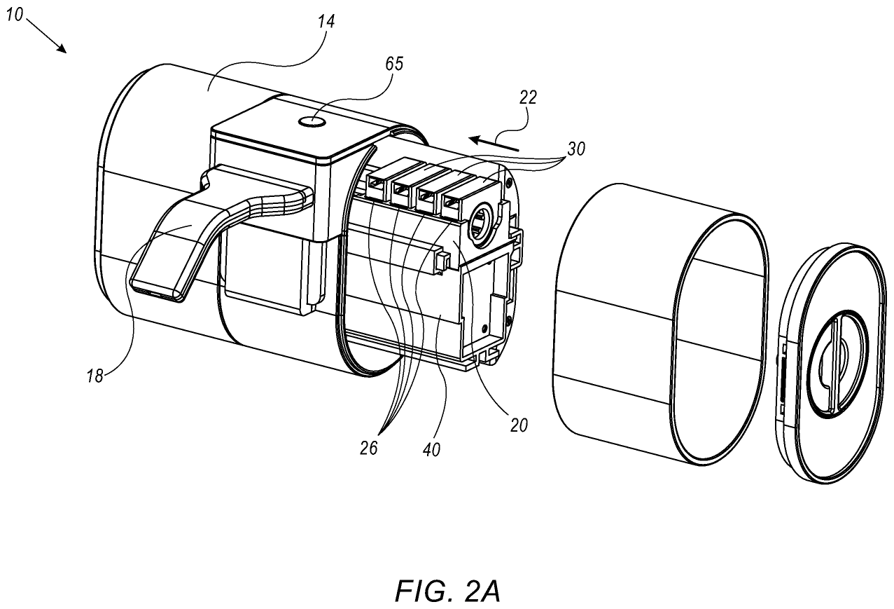

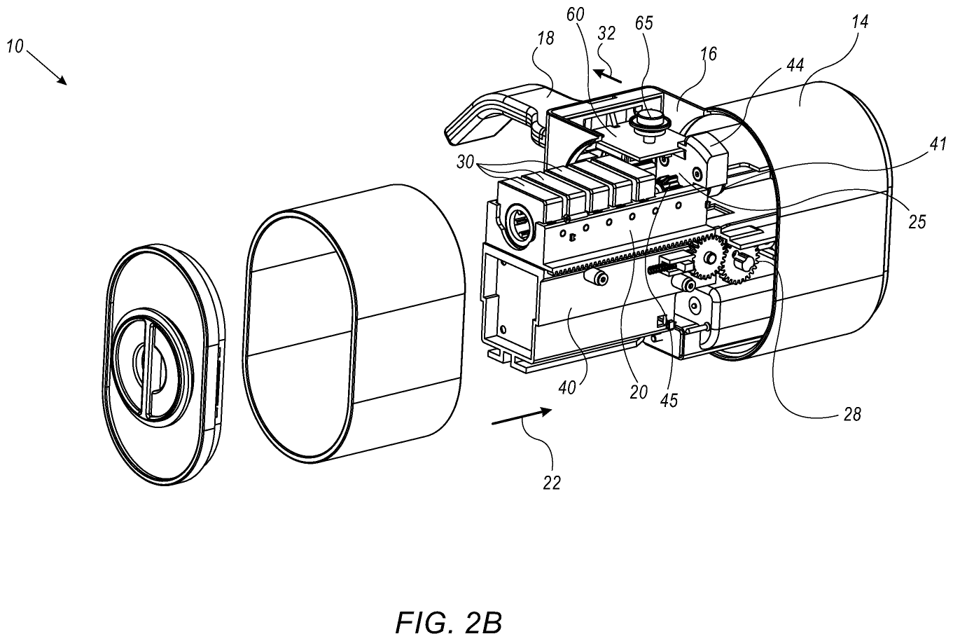

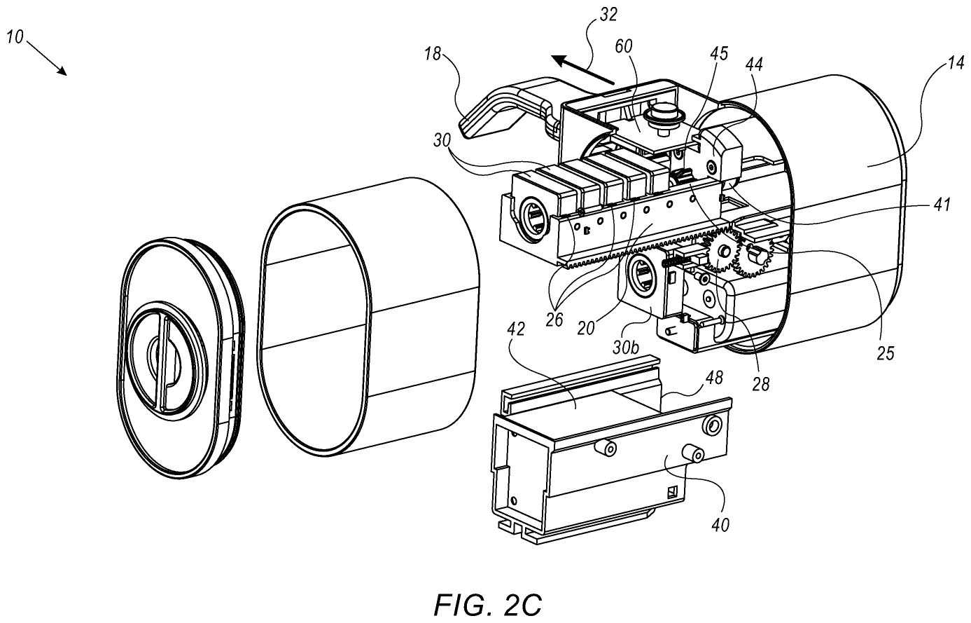

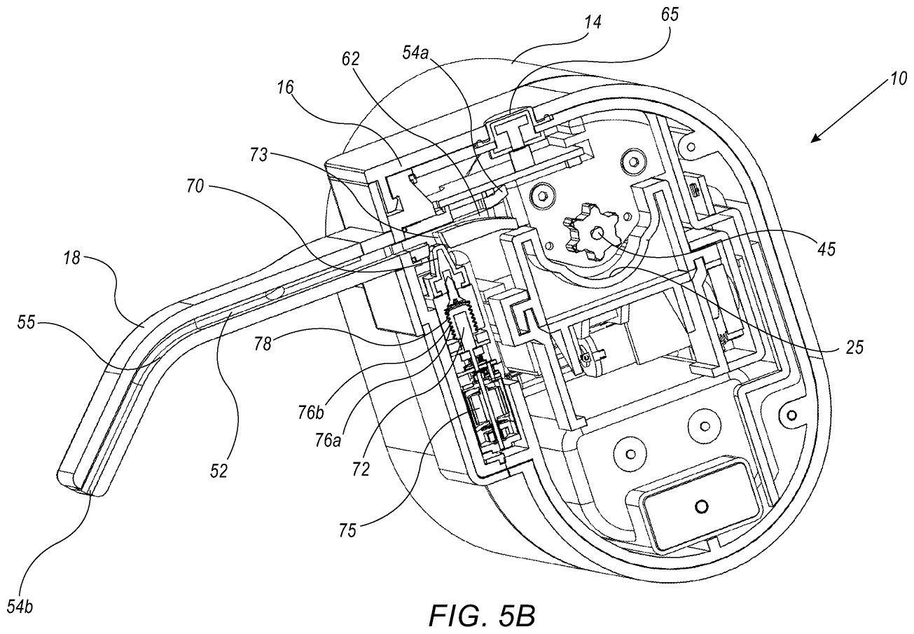

The apparatus 10 further includes a dispenser 16 for dispensing a urine strip 38 disposed in a cartridge 30 towards the urination area and a detector 60 (shown in FIG. 2B) for obtaining data from a used urine strip 38. The dispenser 16 includes a spout 18 extending from the housing 14 towards the urination area and is configured to guide the urine strips 38 from the dispenser 16 towards the testing position, as shown in FIG. 5A. The apparatus 10 can be provided with an actuation button 65, for activation or deactivation of the apparatus.

The housing 14 is configured to hold a plurality of cartridges 30 each of which having urine strips 38 accessible to be extracted by the dispenser 16. As shown in FIGS. 2A-2C, according to an example, a first side of the housing 14, includes a tray 20 configured to hold cartridges 30 arranged along a sliding axis 22. According to the illustrated example the tray 20 is provided with a plurality of holders 26, each of which is configured to hold one cartridge 30. The tray 20 is configured to slide in a direction along the sliding axis 22, towards the dispenser 16, such that one of the cartridges 30 is adjacent the dispenser 16 and the urine strip 38 is available to be extracted. Displacement of the tray 20 can be carried out by means of a sliding motor 28, as can be seen in FIG. 2B.

For sake of clarity, when referring to a cartridge disposed in the extracting spot 25 the cartridge is designated 30a.

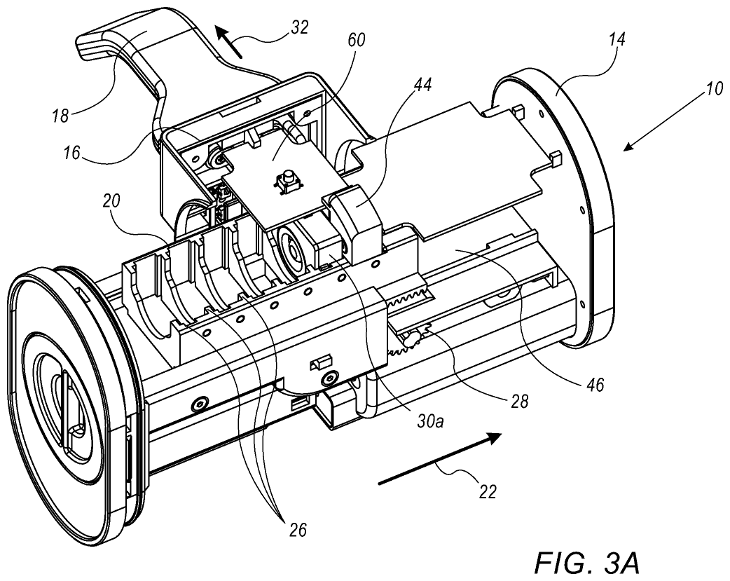

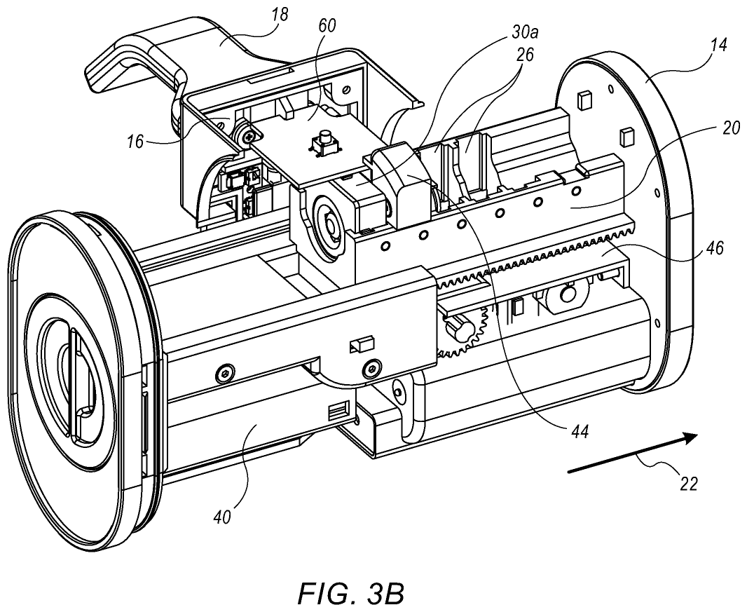

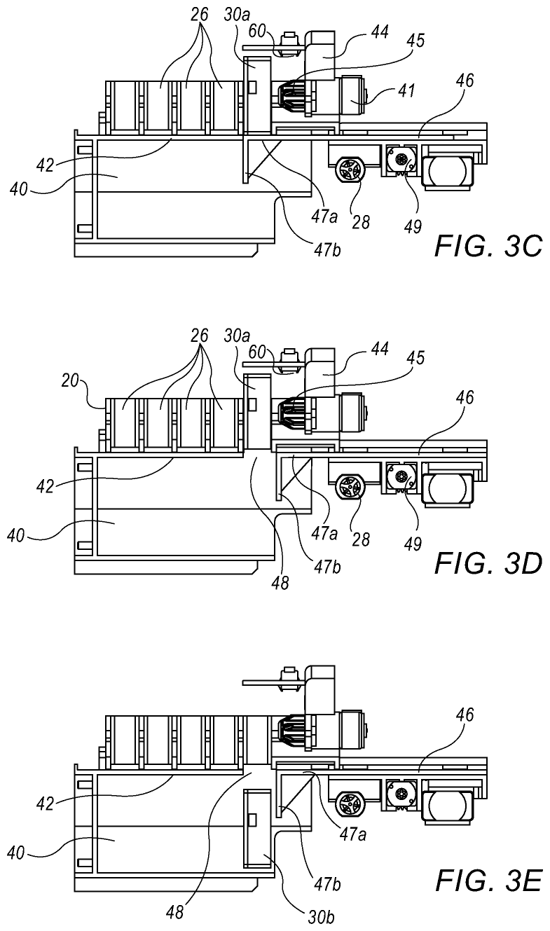

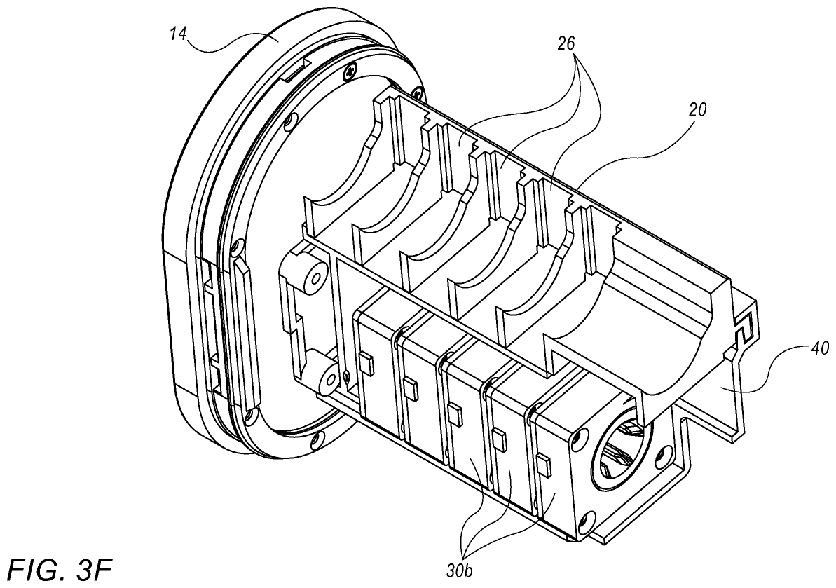

As shown in FIG. 3A, when the tray 20 slides towards the dispenser 16, the first cartridge 30a is positioned in the extracting spot 25, in which the cartridge 30 is disposed along an extracting axis 32. The spout 18 of the dispenser 16 is also disposed along the extracting axis 32 such that, at this extracting spot 25, the strip 38 inside the cartridge 30a can be extracted along the extracting axis 32 from the cartridge 30a into the spout 18.

As can be seen in FIGS. 2B and 2C, the dispenser 16 further includes an extractor 44 for extracting urine strips 38 through spout 18. The extractor 44 includes a cogwheel 45 and an extracting motor 41 configured to rotate the cogwheel 45. The cogwheel 45 extends in the extracting spot 25 along extracting axis 32, facilitating the extraction and retraction of the strip from and to the cartridge 30. Accordingly, while the tray 20 is movable along the sliding axis 22, which is perpendicular to the extraction axis 32.

As explained hereinbelow with reference to FIG. 5A, the extractor 44 is configured to engage the cartridge 30 and to selectively rotate in a first direction and a second direction. Rotation in the first direction urges the urine strip to be extracted out of the cartridge 30 and rotation in the second direction urges the urine strip 38 to be retracted back into the cartridge 30.

Similarly, the detector 60 is disposed such that strip 38 moving along the extracting axis 32 can be detected and analyzed, as explained hereinbelow with reference to FIG. 5B.

The housing 14 further includes a compartment 40 configured to hold used urine strips, i.e., compartment 40 into which the used cartridge 30b are inserted. For sake of clarity, when referring to a used cartridge, which is dropped into compartment 40 the used cartridge is designated 30b.

According to the illustrated example, the compartment 40 is disposed under the tray 20 and includes a top wall 42. According to the illustrated example, the holders 26 of the tray 20 are open from the bottom, such that the cartridges 30 are free to pass into the compartment 40. The top wall 42 of the compartment 40, however holds the cartridges 30 in the holders 26.

The top wall 42 includes an opening 48 located below the extracting spot 25, such that a used cartridge 30b can be dropped from the extracting spot 25 directly into the compartment 40, as shown in FIG. 2C. This way, once the used cartridge 30b is dropped into the compartment 40 the extracting spot 25 is available for the next cartridge 30 in the tray 20 for conducting another urine test.

Referring again to FIGS. 3A and 3B, the tray 20 can further slide towards the dispenser 16 into the extracting position bringing the first cartridge 30 to the extracting spot 25 and causing the extractor 44 to engage the cartridge 30. After the testing of the cartridge is completed, the tray 20 slides back to discarding position such that the cartridge 30a disengages the cogwheel 45 and the cartridge 30a can be removed from the extracting spot 25, in a manner explained in detailed below.

Once the used cartridge 30b is removed, the tray 20 can further slide towards the dispenser 16 bringing the next cartridge to the extracting spot 25. As shown in FIG. 3B. the sliding axis 22 extends between a first side of the extracting spot 25 of the dispenser 16 and a second side thereof. Thus, the tray is slidable under the dispenser 16, such that holders 26 with cartridges 30 are disposed on the first side of the housing and empty holders 26 are disposed on the opposite side of the housing 14. Thus, the tray 20 can slide until the last holder 26 with the last cartridge 30 is brought to the extracting spot 25. At will be appreciated that the tray can be removed from the housing for restacking cartridges 30 in the holders 26 of the tray 20.

The housing 14 further includes a discarding member 46, configured to facilitate moving used cartridge 30b into the compartment 40. As most clearly shown in FIGS. 3C. 3D and 3E, the discarding member 46 includes a horizontal portion 47a, which is movable between a forward position and a backward position with respect to the tray 20 and the top wall 42 of the compartment 40. The discarding member 46 is movable such that in the forward position the end of the horizontal portion 47a covers the opening 48 of the compartment 40.

The discarding member 46 can further include a vertical portion 47b, which extends at the end of the horizontal portion 47a and is configured such that when the discarding member 46 moves towards the compartment 40, the vertical portion 47b pushes the used cartridge 30b into the compartment 40. Thus, when the cartridge 30a is displaced into the extracting spot 25, the horizontal portion 47a of the discarding member 46 is disposed at the forward position, thus covering opening 48 of the compartment 40, and blocking the cartridge 30a from falling into the compartment 40.

As shown in FIG. 3C, once the urine test is completed, the tray 20 slides back to the discarding position, such that the cartridge 30a disengages the cogwheel 45 and the cartridge 30a can be removed. As shown, at this stage the horizontal portion 47a of the discarding member 46 is still disposed at the forward position, thus covering opening 48 of the compartment 40, and blocking the cartridge 30a from falling into the compartment 40.

However, as shown in FIG. 3D, the discarding member 46 is displaced into the backward position thereof, allowing the used cartridge 30b to drop through opening 48 into the compartment 40. As shown in FIG. 3E, when the used cartridge 30b reaches the compartment 40, the discarding member 46 is displaced back to the forward position, and the vertical portion 47b pushes the used cartridge 30b into the inner portion of the compartment 40. This way, the area of the compartment 40 below the opening 48 is available for the next used cartridge 30b. As shown in FIG. 3F, the compartment 40 can thus be filled with a plurality of used cartridges 30b. Displacement of the discarding member 46 between the forward and backward positions can be carried out by a discarding motor 49.

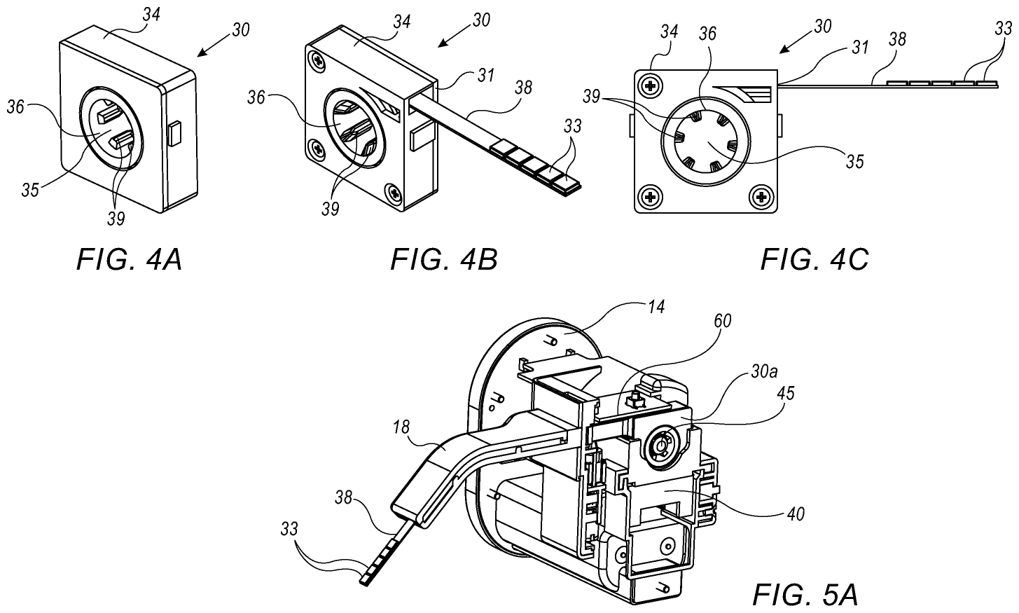

With reference to FIGS. 4A-4C, the cartridge 30 can include a case 34 and a reel 36 mounted inside the casing and is configured to allow winding a urine strip 38 thereon. The reel 36 is provided with an aperture 35 and a plurality of gear teeth 39, corresponding to the cogwheel 45, such that the reel 36 can be rotated by the cogwheel 45. As shown in FIGS. 4B and 4C, the case 34 is configured to encase the urine strip 38 wounded on the reel 36, and includes an opening 31 through which the urine strip 38 can be extracted out of the cartridge 30. This way, the rotation of cogwheel 45 in a first direction causes the reel 36 to rotate in the same direction urging thereby the urine strip 38 to be extracted out of the cartridge 30. Rotation of cogwheel 45 in a second direction causes the reel 36 to rotate in the same direction urging, thereby the urine strip 38 to be retracted back into the cartridge 30.

The urine strip 38 can include a plurality of testing pads 33 each of which is impregnated with chemicals that react with a compound present in urine, such as pH level, Ketone, Haemoglobin, etc. The testing pads 33 can be disposed along the strip such that when the strip 38 is extracted from the cartridge 30 the testing pads 33 are disposed out of the cartridge 30.

It will be appreciated that the length at which the urine strip 38 is extracted out of the cartridge 30 can be determined in accordance with the length of the spout 18 and the intended use of the urine strip 38. For example, the length of the urine strip can be configured to accommodate male users or female user, so that the extracted urine strip 38 is disposed along the user's urine path.

Moreover, according to an example each cartridge 30 can include a plurality of urine strips 38 wounded thereon. The cartridge 30 can be configured such that used urine strips 38 retracted back into the case 34 are wounded or stored such that the next strip on the reel 36 is available to be extracted.

As shown in FIGS. 5A and 5B, when the cartridge 30a is displaced by sliding tray 20 into the extracting spot 25 the cogwheel 45 engages the gear teeth 39 of reel 36. Rotation of the cogwheel 45 in one direction causes the strip 38 to be extracted out of the cartridge 30a into the spout 18 along the extracting axis 32. The spout 18 includes a channel 52 extending between an inlet 54a, disposed adjacent the opening 31 of the cartridge 30a, and an outlet 54b, at the end of the spout 18. The channel 52 is configured with a size and a shape to support the urine strip 38 and can define a curved path 55 between the inlet 54a and the outlet 54b. For example, the curved path 55 can be configured such that the outlet 54a is disposed inside the urination device and the urine strip 38 extracted from the cartridge 30a is disposed in the desired location in the urination area. This way, the spout 18 brings the urine strip 38 to the urination position, along the expected path of the urine.

It will be appreciated that the curved path 55 can include various curves in accordance with the size and shape of the urination device and its intended use. As indicated above the length of the urine strip can be configured to accommodate male users or female user, so that the extracted urine strip 38 is disposed along the user's urine path. Alternatively, the spout 18 can be configured to accommodate certain users, such as males or females, so that the extracted urine strip 38 is disposed along the user's urine path. Similarly, the spout 18 can be configured to accommodate users of certain ages or body sizes.

As shown in FIGS. 5A and 5B, the dispenser 16 defines a testing spot 62 along the extracting axis 32 such that the detector 60 can obtain images of the testing pads 33 passing through the testing spot 62. For example, the testing spot 62 can be defined inside the channel 52, and the detector 60 is disposed above the testing spot 62 such that images of the used strip 38 can be captured, while it is retracted back into the cartridge 30a.

According to an example, the detector 60 is configured to obtain a series of images of each of the testing pads 33 on the retracted strip 38. The detector 60 and the cogwheel 45 can be synchronized such that the retraction of the strip allows obtaining images of each of the testing pads. For example, the speed of the rotation of the cogwheel 45 can be such which allows the detector to obtain at least one image of each of the testing pads 33. According to another example, the cogwheel 45 can be configured to rotate in steps, such that while the strip 38 is retracted, the displacement of the testing pads 33 stops while each of the pads is at the testing spot 62, allowing the detector 60 to obtain an image of each of the pads 33. According to the illustrated example the testing pads 33 are arranged along the length of the strip 38, such that along the width thereof there is a single testing pad 33. This allows the detector 60 to obtain an image of each testing pad 33 separately.

According to other examples, the strip may include more than one testing pad 33 that along the width thereof, and the detector 60 can be configured to obtain an image of more than one testing pad 33 at a time. According to this example, information from the obtained image can be analyzed in accordance with the disposition of each of the testing pad 33 with respect to the strip 38.

As shown in FIG. 5B, the dispenser 16 can further include a seal 70 configured to seal the channel 52, when the apparatus 10 is not in use. The seal 70 is configured to block moisture from entering the tray 20 and damaging the urine strips 38. According to the illustrated example, the seal is movable into the channel 52 via a sealing opening 73. The seal 70 can be actuated by an actuator, here illustrated as a motor 75 and a motion converter which is configured to convert rotational motion of the motor into a linear motion, such that rotation of the motor 75 urges the seal 70 in or out of the opening 73.

According to the illustrated example, the motion converter includes a shaft 72 mounted on the motor and having a screw thread 76a, and a bolt 78 having a corresponding screw thread 76b. The seal 70 is mounted on the bolt 78 such that rotation of the motor 75 and the shaft 72 in a first direction urges the bolt 78 and the seal 70 to move forward into the channel 52. Rotation of the motor 75 and the shaft 72 in a second direction urges the bolt 78 and the seal 70 to move backwards out of the channel 52, allowing a urine strip 38 to move through the channel 52.

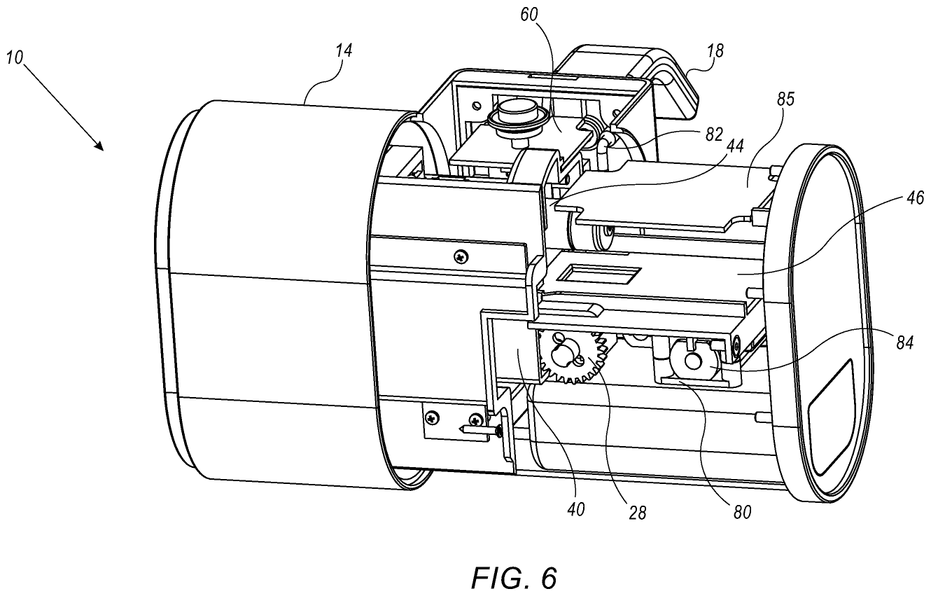

Referring now to FIG. 6, the apparatus can further include a cleaning mechanism 80, for clearing the channel 52 of any residues of urine. The cleaning mechanism 80 can include an air pump 84 and a pipe 82 extending between the channel 52 and the air pump 84. When the cleaning mechanism 80 is activated, airflow generated by the air pump 84 forces any remaining urine out of the channel towards the outlet 54b and into the urination device. The channel 52 can include a hydrophobic surface, repelling any remaining urine such that together with the cleaning mechanism 80 remaining urine is urged out of the spout 18 and into the toilet.

On the other hand, the air pump 84 sucks air from the inner space of the housing 14, thus creating vacuum in the housing. The vacuum in the housing is further facilitated by the seal 70 which blocks any intake of air through the channel 52. The degree of vacuum formed inside the housing is such that optimizes the maintenance of the urine strips 38 stored in the housing 14.

Moreover, in order to further maintain the apparatus 10, the spout 18 can be provided with a UV source configured for to provide the channel 52 with ultraviolet radiation disinfecting thereby any remaining urine in the channel 52.

The apparatus can further include a controller 85 configured to control the operation of the sliding tray 20 towards the extracting spot 25, the operation of the extractor 44 urging the urine strip in and out of the spout 18. The controller 85 is further configured to control the operation of the detector 60 and can include a processor for processing the images obtained by the detector 60. In addition, the controller 85 can be configured to control the displacement of the discarding member 46 between the forward and backward positions, Finally, the controller 85 can be configured to control the operation of the seal 70 in and out of the channel 52, and the operation of the cleaning mechanism 80.

According to an example, the controller 85 is provided with a wireless communication module, such that the control can send and receive data to a mobile device, for example a smart phone. The mobile device can be provided with a designated software application which provides the user with data related to the apparatus 10. For example, the controller 85 can be configured to send the mobile device data related to the results of the urine tests. The controller 85 can also be configured to send the mobile device information regarding the state of the apparatus. For instance, in case all the cartridges 30 are used and the tray 20 needs to be restacked, the user can receive an indication. The mobile device maybe further configured to control the operation of the apparatus, such as turning the apparatus on or off or controlling the timing at which the urine tests will be conducted.

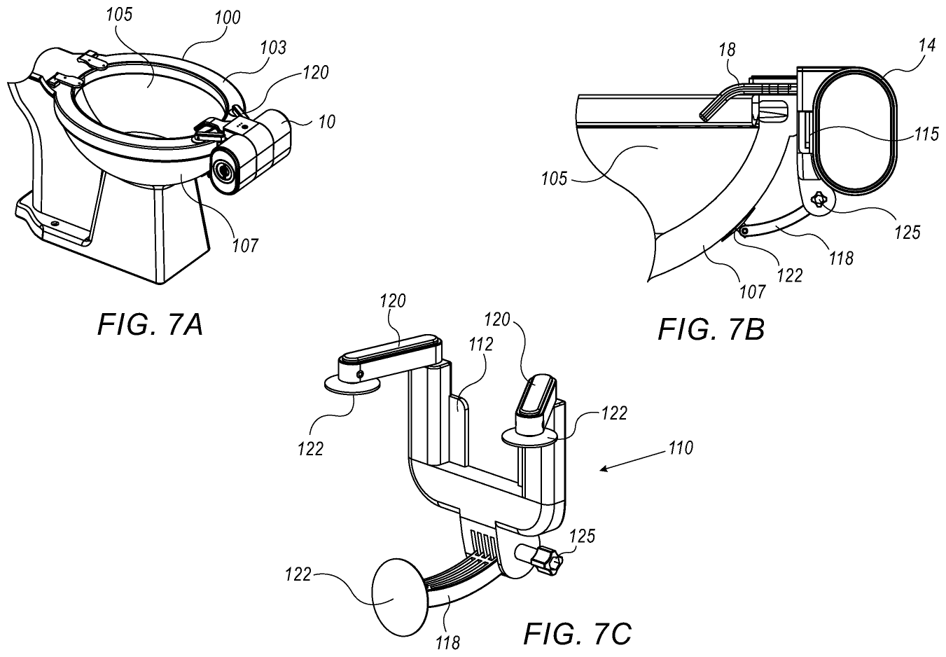

Referring now to FIGS. 7A-7C, the apparatus 10 can be mounted on a urination device, such as a toilet 100, such that the housing 14 is disposed out of the toilet bowl 105 while the spout 18 extends inside the toilet bowl. According to the illustrated example, the apparatus 10 includes a mounting member 110 configured to mount the housing 14 to the outer wall 107 of the toilet bowl 105. The mounting member 110 includes a seat 112 for holding a corresponding flange 115 (most clearly shown in FIG. 1), defined on the housing 14. The flange 115 can be defined below the spout 18, such that when the flange is held in the seat 112 of the mounting member 110, the housing can be mounted on the outer wall 107 of the toilet bowl and the spout 18 extends into the toilet bowl.

According to an example, the mounting member 110 includes a bottom arm 118 configured to be affixed to the outer wall 107, and one or more top arms 120 configured to be affixed to the top lip 103 of the toilet bowl, the mounting member 110 further include an adjusting pivot 125 which allows adjusting the angle between the bottom arm 118 and the top arms 120. This way, the mounting member 110 is a universal mounting member which can be mounted on a toilet of any shape and size.

The bottom arm 118 and the top arms 120 can be provided with a suction cup 122, for affixing the arms to the outer wall 107 and top lip 103 of the toilet. The suction cups 122 are configured to provide a stable mounting of the mounting member 110 to the toilet bowl, however, allow removing the mounting member 110, for example, for replacing the cartridges 30 in the tray 20.

Those skilled in the art to which the presently disclosed subject matter pertains will readily appreciate that numerous changes, variations, and modifications can be made without departing from the scope of the invention, mutatis mutandis.

1. An apparatus for conducting urinalysis of urine, the apparatus comprising:
a housing mountable on a urination device defining a urination area;
a tray for holding a plurality of cartridges, each of which including a urine strip having testing pads, said tray is movable inside said housing and is configured to displace at least one cartridge toward an extracting spot;
a dispenser configured for extracting said urine strip out of said cartridge and conveying said testing pads into said urination area along an extraction path extending between said extracting spot and said urination area; and
a detector configured to obtain data from said testing pads when said testing pads are transferred along said extraction path.

2. The apparatus of claim 1 wherein said dispenser includes a spout defining a channel extending between said extracting spot inside said housing and said urination area.

3. The apparatus of claim 1 wherein said tray is movable along a sliding axis, wherein said sliding axis is perpendicular to said extraction path, and extend between a first side of said dispenser and a second side of said dispenser.

4. The apparatus of claim 1 wherein said dispenser includes an extractor configured to engage said cartridge and to selectively rotate in a first direction and a second direction, wherein rotation in said first direction urges the urine strip to be extracted out of the cartridge and rotation in said second direction urges the urine strip to be retracted back into the cartridge.

5. The apparatus of claim 4 wherein said cartridge includes a case and a reel rotatably mounted inside the case, said reel is configured to allow winding said urine strip thereon, and is configured such that rotation thereof extracts said urine strip out of said case.

6. The apparatus of claim 5 wherein said reel includes an aperture and a plurality of gear teeth, and said extractor includes a cogwheel configured to engage said plurality of gear teeth and to rotate said reel about an axis perpendicular to said extraction path.

7. The apparatus of claim 1 further comprising a compartment configured to hold used cartridge, said compartment includes an opening disposed adjacent said extracting spot.

8. The apparatus of claim 7 further comprising a discarding member movable with respect to said extracting spot between a forward position and a backward position, wherein in said forward position a horizontal portion of said discarding member covers the opening of the compartment, and wherein in said backward position said opening allows discarding said cartridge from said tray into said compartment.

9. The apparatus of claim 8 wherein said discarding member further includes a vertical portion configured such that in said forward position said vertical portion protrudes into said compartment, pushing thereby cartridges disposed below said opening toward an inner section of said compartment.

10. The apparatus of claim 2 wherein said dispenser defines a testing spot along the extracting axis and the detector is disposed at said testing spot.

11. The apparatus of claim 10 wherein said testing spot is defined inside said and said detector is configured to obtain an image of the testing pads while the urine strip is retracted back into the cartridge.

12. The apparatus of claim 2 wherein said dispenser includes a seal configured to displace in a first direction in which the seal is urged into the channel and a second direction in which the seal is displaced out of the channel allowing said urine strip to move through the channel.

13. The apparatus of claim 2 further comprising a cleaning mechanism configured for clearing the channel of any residues of urine.

14. The apparatus of claim 13 wherein said cleaning mechanism includes an air pump configured to generate an airflow inside said channel forcing any remaining urine out of the channel towards the urination area.

15. The apparatus of claim 14 wherein said air pump is further configured to suck air from an inner space of the housing creating thereby vacuum in the housing.

16. The apparatus of claim 2 wherein channel includes a hydrophobic surface, repelling any remaining urine.

17. The apparatus of claim 1 further comprising a mounting member configured to mount the housing to an outer wall of a urination device.

18. The apparatus of claim 17 wherein said mounting member includes a seat for holding a corresponding flange defined on the housing.

19. The apparatus of claim 17 wherein said mounting member include a bottom arm configured to be affixed to said outer wall, and a least one top arm configured to be affixed to a top lip of the urination device.

20. The apparatus of claim 19 wherein said mounting member further include an adjusting pivot configured for adjusting an angle between said bottom arm and said at least one top arm.
